(19) United States
(12) Patent Application Publication
(10) Pub. No.: US 2010/0292788 A1
(43) Pub. Date: Nov. 18, 2010

(54) UNITARY SCLERAL BUCKLING ELEMENT AND METHODS OF USING SAME

(76) Inventor: Robert E. Morris, Birmingham, AL (US)

Correspondence Address: Ballard Spahr LLP, SUITE 1000, 999 PEACHTREE STREET, ATLANTA, GA, 30309-3915, US

(21) Appl. No.: 12/768,417

(22) Filed: Apr. 27, 2010

Related U.S. Application Data
(60) Provisional application No. 61/173,015, filed on Apr. 27, 2009.

(51) Int. Cl. A61F 2/14

(52) U.S. Cl. 623/4.1

(57) ABSTRACT

A unitary scleral buckling element for treating a retinal defect within an eye of a subject is disclosed. The scleral buckling element has a band portion for encircling the eye and an enlarged portion formed along the length of the band portion, which is configured to overlie a retinal defect. In one aspect, the width of the enlarged portion is greater than the width of the band portion of the scleral buckling element.

### CROSS-REFERENCE TO RELATED PATENT APPLICATION

This application claims the benefit of U.S. Provisional Application No. 61/173,015, filed on Apr. 27, 2009, which is hereby incorporated by reference in its entirety.

### FIELD OF THE INVENTION

This invention relates to a scleral buckling element and, more specifically, to a single-piece scleral buckling element having an enlarged portion for overlying and supporting a retinal defect in an eye of a subject.

### BACKGROUND

Conventionally, a rhegmatogenous retinal detachment in an eye of a subject can be treated by “buckling” the scleral wall of the eye inwardly to contact the retina such that retinal defects, including retinal holes and tears, are closed. Scleral buckling can be accomplished using encircling buckles, which conventionally include a narrow band overlying a segmental “tire” buckle (“TB”) as depicted in FIG. 1. The tire buckle is conventionally of greater width and thickness than the narrow band of the encircling buckle. As depicted in FIGS. 2A-2B, the tire buckle conventionally has a central groove for receiving the narrow band of the encircling buckle.

During a conventional scleral buckling procedure, the narrow band is initially placed around the eye of a subject. The ends of the narrow band are attached to each other, and the excess portions of the band are trimmed to ensure that the surfaces of the band are smooth. The tire buckle is then re-sized as necessary such that the size of the tire buckle corresponds to the size of the retinal defect. The tire buckle is subsequently placed underneath the narrow band such that the tire buckle overlies the retinal defect. The tire buckle is then attached to the eye wall using scleral sutures such that the tire buckle is enfolded into the eye wall as shown in FIG. 1. The narrow band is then anchored to other portions of the eye using sutures. Finally, the band is tightened around the eye in a desired position, and the attachment between the ends of the narrow band is permanently set.

As depicted in FIG. 2A, the tire buckle is typically supplied as a circular ring of silicone rubber that is laterally cut to produce a linear tire section. Then, the linear tire section is cut to a desired length. In practice, the linear tire section must have an appropriate length or this process will have to be repeated. After the tire buckle is sized, the tire buckle is placed against the eye of the subject to confirm that the size of the tire buckle is appropriate for overlying the retinal defect in the eye. The size of the tire buckle is then adjusted as necessary, and edges produced during the re-sizing of the tire buckle must be trimmed to create smooth surfaces. Failure to smoothly trim the edges of the tire buckle can ultimately lead to erosion of the scleral wall following implantation of the encircling buckle into the eye of the subject. This erosion of the scleral wall can cause intraocular hemorrhage and/or require buckle removal surgery. After the tire buckle is adequately trimmed and sized, the tire buckle is positioned underneath the narrow band such that the tire buckle overlies the retinal defect. The entire process of cutting, sizing, trimming, placing, and suturing the tire buckle must be accomplished during the surgical procedure while the subject is under anesthesia. Thus, the preparation and attachment of the tire buckle can significantly extend the duration of surgery.

Accordingly, there is a need in the pertinent art for a scleral buckling element that reduces the total amount of time required to perform a scleral buckling procedure. Specifically, there is a need in the pertinent art for a unitary scleral buckling element that is pre-configured for implantation around the eye. There is a further need in the pertinent art for a scleral buckling element that decreases the likelihood of scleral wall erosion in an eye of a subject following a scleral buckling procedure.

### SUMMARY

Described herein is a scleral buckling element for treating retinal defects within an eye of a subject. The scleral buckling element has a band portion for encircling the eye and an enlarged portion formed along the length of the band portion. The enlarged portion is configured to overlie a retinal defect. The width of the enlarged portion is greater than the width of the band portion of the scleral buckling element. The enlarged portion and the band portion of the scleral buckling element are of unitary construction. The enlarged portion and the band portion of the scleral buckling element are also equally compressive. The enlarged portion and the band portion of the scleral buckling element also have a uniform thickness. The length of the enlarged portion can correspond to the length of one or more quadrants of the eye of a subject. The outer edges of the enlarged portion can define a variety of shapes, and the outer edges of the enlarged portion can be smooth. Methods of treating retinal defects within an eye of a subject using the scleral buckling element are also described.

### BRIEF DESCRIPTION OF THE FIGURES

These and other features of the preferred embodiments of the invention will become more apparent in the detailed description in which reference is made to the appended drawings wherein:

FIG. 1 is a side perspective view of a conventional encircling buckle positioned on an eye of a subject.

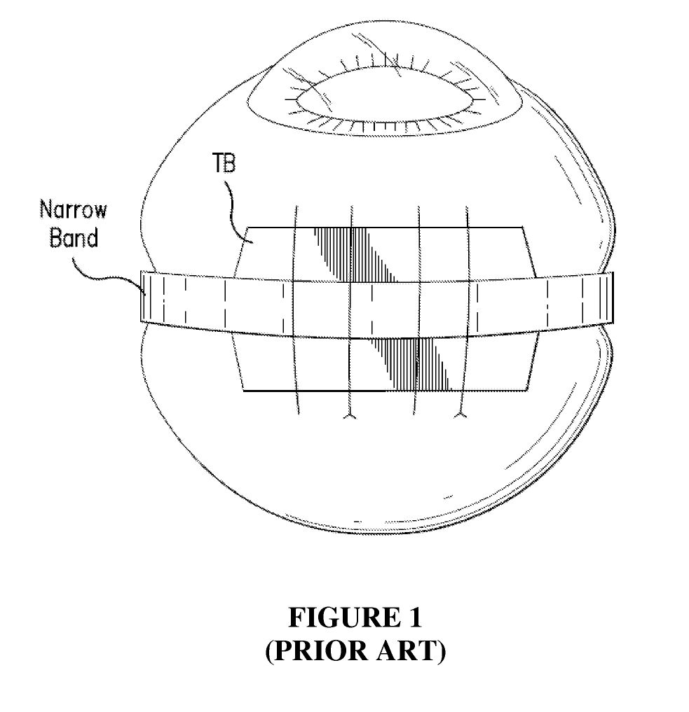

FIG. 2A is a perspective view of a conventional tire buckle prior to lateral cutting of the tire buckle. FIG. 2B is a cross-sectional profile view of the conventional tire buckle depicted in FIG. 2A.

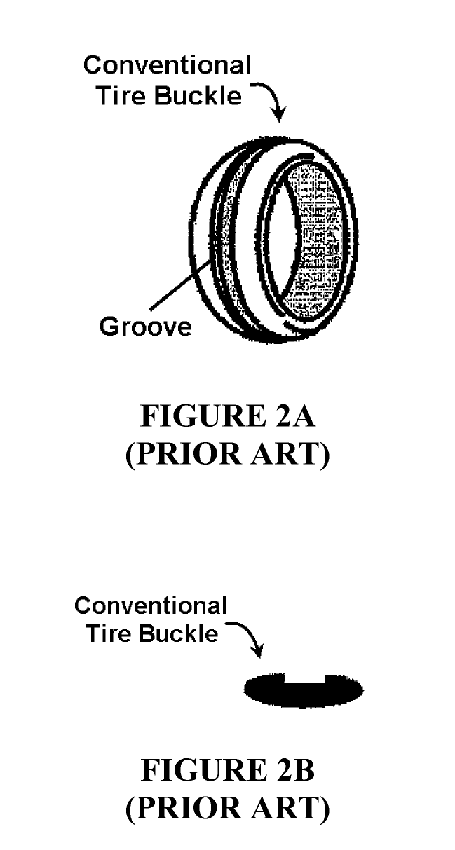

FIG. 3 is a side perspective view of a scleral buckling element as described herein positioned on an eye of a subject.

FIG. 4 includes side perspective views of exemplary scleral buckling elements as described herein.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a retinal defect” can include two or more such retinal defects unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

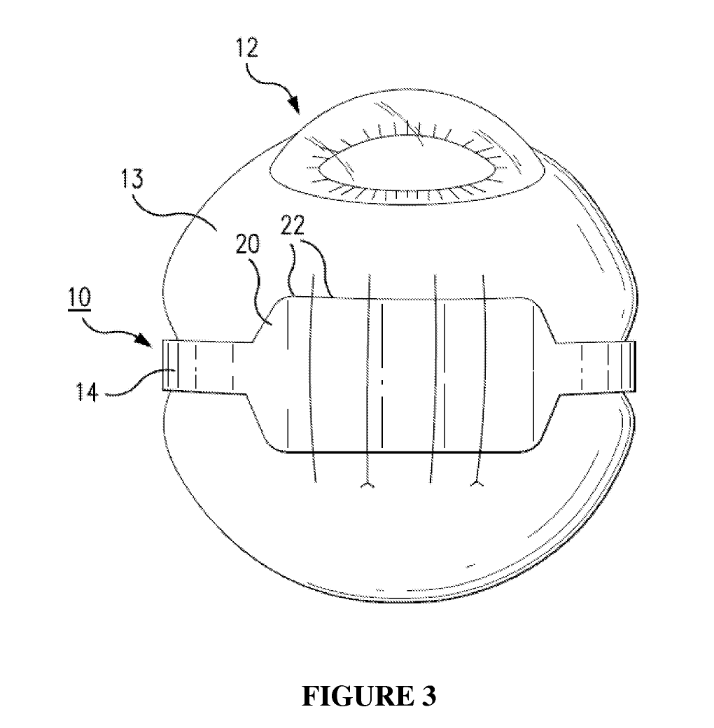

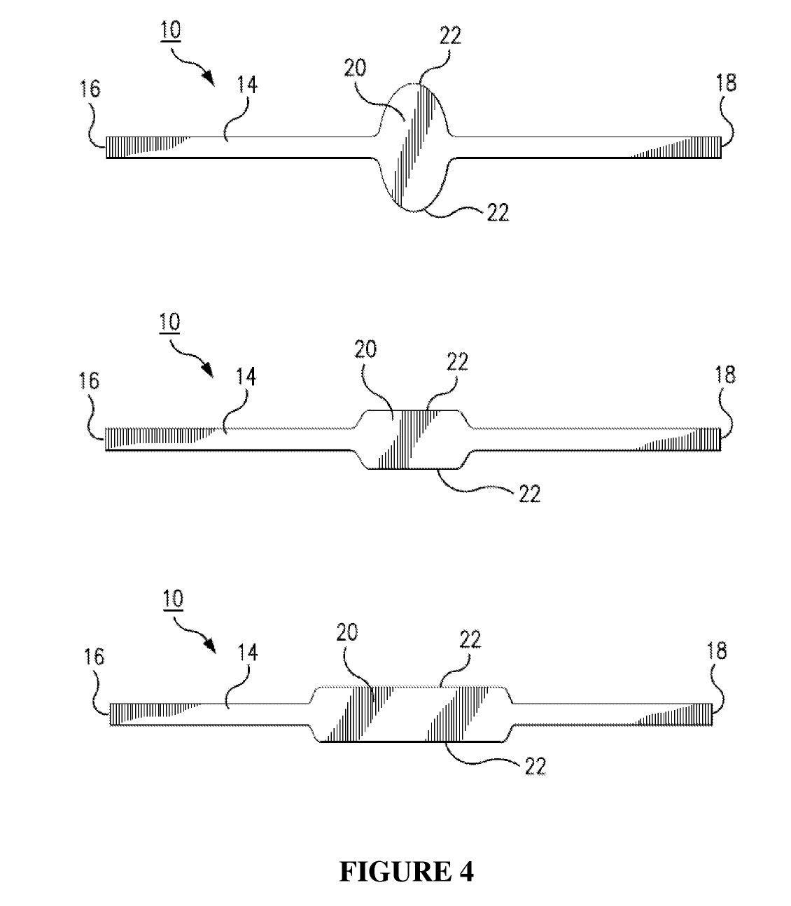

In one embodiment, and with reference to FIGS. 3 and 4, the invention relates to a unitary scleral buckling element 10 for treating a retinal defect within an eye 12 of a subject. In one aspect, the scleral buckling element 10 can comprise a band portion 14 having a length and a width. In this aspect, the length of the band portion 14 can be configured to encircle the eye 12 of the subject. It is contemplated that the length of the band portion 14 can be configured to substantially equatorially encircle the eye 12 of the subject. Accordingly, in one non-limiting example, the length of the band portion 14 can range from between about 60 mm to about 90 mm.

In one aspect, the band portion 14 of the scleral buckling element 10 can comprise a first end 16 and a second end 18 as depicted in FIG. 4. In this aspect, the first end 16 and the second end 18 of the band portion 14 can be configured for attachment to one another such that the band portion can encircle the eye 12 of the subject as depicted in FIG. 3. It is contemplated that the first end 16 of the band portion 14 can be attached to the second end 18 of the band portion using at least one of a conventional suture or a conventional sleeve.

In another aspect, the scleral buckling element 10 can comprise an enlarged portion 20 formed along a portion of the length of the band portion 14. In this aspect, it is contemplated that the enlarged portion 20 of the scleral buckling element 10 can be positioned centrally along the length of the band portion 14. In an additional aspect, the enlarged portion 20 of the scleral buckling element 10 can have a length and a width. In this aspect, it is contemplated that the width of the enlarged portion 20 can be greater than the width of the band portion 14. It is further contemplated that the width of the enlarged portion 20 can vary along the length of the enlarged portion. In an additional aspect, the width of the enlarged portion 20 of the scleral buckling element 10 can range from about 2.5 mm to about 10 mm.

In another aspect, the enlarged portion 20 of the scleral buckling element 10 can be configured to substantially overlie the retinal defect in the eye 12 of the subject. In a further aspect, when the retinal defect in the eye 12 of the subject comprises a radial, horseshoe-shaped retinal tear, the width of the enlarged portion 20 of the scleral buckling element 10 can range from about 20 mm to about 25 mm. In still a further aspect, the width of the band portion 14 can range from about 2 mm to about 3 mm. It is contemplated that the ratio between the width of the enlarged portion 20 and the width of the band portion 14 can range from about 1:1 to about 8:1. For example, the ratio between the width of the enlarged portion 20 and the width of the band portion 14 can be 1:1, 1.5:1, 2:1, 2.5:1, 3:1, 3.5:1, 4:1, 4.5:1, 5:1, 5.5:1, 6:1, 6.5:1, 7:1, 7.5:1, 8:1, and all ratios in between.

In an additional aspect, the enlarged portion 20 and the band portion 14 of the scleral buckling element 10 can be a unitary or otherwise monolithic construction. Thus, it is contemplated that the enlarged portion 20 and the band portion 14 of the scleral buckling element 10 can comprise a single piece of material having uniform properties along the lengths of the band portion and the enlarged portion. For example, and without limitation, the enlarged portion 20 and the band portion 14 of the scleral buckling element 10 can be equally compressive.

In another aspect, the band portion 14 and the enlarged portion 20 of the scleral buckling element 10 can each have a thickness. In one aspect, the enlarged portion 20 and the band portion 14 can have a substantially uniform thickness. In this aspect, it is contemplated that the term “substantially uniform thickness” can indicate that the thickness of the enlarged portion 20 can deviate from the thickness of the band portion 14 within a functionally insignificant range. However, it is further contemplated that the term “substantially uniform thickness” can indicate that the thickness of the enlarged portion 20 is equal to the thickness of the band portion 14.

In a further aspect, and with reference to FIG. 3, the band portion 14 and the enlarged portion 20 of the scleral buckling element 10 can be configured to be securely positioned on the scleral wall 13 of the eye 12 of the subject such that the enlarged portion overlies and supports the retinal defect. In this aspect, it is contemplated that the scleral buckling element 10 can be tightened around the eye 12 of the subject such that the enlarged portion 20 applies pressure to a portion of the scleral wall 13 of the eye 12 overlying the retinal defect, thereby forcing the scleral wall inwardly such that the retinal defect is positioned against the scleral wall.

In one aspect, the retinal defect can comprise a detached retina. In this aspect, it is contemplated that the detached retina can be caused by, for example and without limitation, at least one of a retinal hole, a retinal tear, and a separation of a sensory retina layer of the eye of the subject from a retinal pigment epithelium layer of the eye of the subject.

It is further contemplated that the scleral wall 13 can be forced inwardly such that fluid within the retinal defect is cleared and the retinal defect is closed against the inwardly buckled eye wall. It is still further contemplated that positioning of the scleral wall 13 against a detached retina can close the retinal hole, retinal tear, and/or other separation causing the detached retina, thereby promoting re-attachment of the retina. When the retinal defect comprises a radial, horseshoe-shaped tear, it is contemplated that the scleral buckling element 10 can be securely positioned on the scleral wall 13 of the eye 12 of the subject using sutures placed radially relative to the band portion 14 of the scleral buckling element.

In an additional aspect, the eye 12 of the subject can comprise four quadrants of equal length. In another aspect, the length of the enlarged portion 20 of the scleral buckling element 10 can substantially correspond to the length of one quadrant of the eye 12 of the subject. In this aspect, it is contemplated that the length of the enlarged portion 20 of the scleral buckling element 10 can range from about 15 mm to about 25 mm.

In a further aspect, the length of the enlarged portion 20 of the scleral buckling element 10 can substantially correspond to the length of two quadrants of the eye 12 of the subject. In this aspect, it is contemplated that the length of the enlarged portion 20 of the scleral buckling element 10 can range from about 30 mm to about 50 mm.

In still a further aspect, the length of the enlarged portion 20 of the scleral buckling element 10 can substantially correspond to the length of three quadrants of the eye 12 of the subject. In this aspect, it is contemplated that the length of the enlarged portion 20 of the scleral buckling element 10 can range from about 45 mm to about 75 mm. However, it is further contemplated that the length of the enlarged portion 20 of the scleral buckling element 10 can correspond to the size of any potential retinal defect within the eye 12 of the subject. Accordingly, it is contemplated that the length of the enlarged portion 20 of the scleral buckling element 10 can be greater or less than the ranges specified above.

In a further aspect, the enlarged portion 20 of the scleral buckling element 10 can have outer edges 22 that cooperate to define a shape of the enlarged portion. In this aspect, it is contemplated that the shape of the enlarged portion 20 can be, for example and without limitation, substantially circular, substantially elliptical, substantially rectangular, and the like. It is further contemplated that the outer edges 22 of the enlarged portion 20 of the scleral buckling element 10 can be substantially smooth. It is still further contemplated that the smooth outer edges 22 of the enlarged portion 20 can decrease the likelihood of scleral wall erosion within the eye 12 of the subject following implantation of the scleral buckling element 10.

In another aspect, the scleral buckling element 10 can comprise at least one of a conventional elastomeric material, a conventional rubber material, and a conventional plastic material. For example, and without limitation, the scleral buckling element 10 can comprise silicone.

In use, the scleral buckling elements described herein can be employed in a method for treating the retinal defect within the eye of the subject. In one aspect, the method for treating the retinal defect can comprise substantially equatorially encircling the eye of the subject with a scleral buckling element as described herein. In another aspect, the method for treating the retinal defect can comprise securely attaching a scleral buckling element as described herein to the eye of the subject such that the enlarged portion of the scleral buckling element overlies and supports the retinal defect.

It is contemplated that the step of substantially encircling the eye of the subject with the scleral buckling element can comprise making a 360 degree conjunctival peritomy and isolating the four rectus muscles prior to the step of equatorially encircling the eye of the subject with the scleral buckling element. Following isolation of the rectus muscles, the step of substantially encircling the eye of the subject with the scleral buckling element can further comprise passing the scleral buckling element under each rectus muscle such that the wide portion of the scleral buckling element is positioned in the quadrant or quadrants wherein the retinal defect has been identified by a medical practitioner, such as, for example and without limitation, a retinal surgeon.

In between the steps of substantially encircling the eye of the subject with the scleral buckling element and securely attaching the scleral buckling element, it is contemplated that the method for treating the retinal defect can comprise treating an edge of the retinal defect with at least one of cryopexy, laser, endolaser, and diathermy to effect adhesion of the sensory retina to the apposing pigment epithelium of the retina.

It is further contemplated that the step of securely attaching the scleral buckling element can comprise suturing the scleral buckling element to the scleral wall. The step of securely attaching the scleral buckling element can further comprise pulling the respective ends of the scleral buckling element to tighten the the scleral buckling element around the eye of the subject, thereby increasing the application of pressure to the portion of the scleral wall overlying the retinal defect.

In an additional aspect, after the step of securely attaching the scleral buckling element, the method for treating the retinal defect within the eye of the subject can comprise trimming the ends of the scleral buckling element such that the ends are substantially smooth. In a further aspect, after the step of securely attaching the scleral buckling element, and upon completion of the surgical procedure, the conjunctiva of the eye of the subject can be closed.

It is contemplated that the steps of encircling the eye of the subject with a scleral buckling element and securely attaching the scleral buckling element can be completed without any alteration of the size, shape, and/or profile of the scleral buckling element. It is further contemplated that the scleral buckling elements as described herein can be identified and classified according to, for example and without limitation, the width of the enlarged portion, the length of the enlarged portion, the shape of the enlarged portion, and the ratio of the width of the enlarged portion to the width of the band portion. Thus, it is contemplated that a medical practitioner, such as, for example and without limitation, a retinal surgeon, can efficiently identify and select an appropriate scleral buckling element for use in a scleral buckling procedure. After selecting the appropriate scleral buckling element, it is contemplated that the medical practioner can implant the scleral buckling element without making any adjustments to the size, shape, and/or profile of the scleral buckling element.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A unitary scleral buckling element for treating a retinal defect within an eye of a subject, comprising:
a band portion having a length and a width, wherein the elongate length of the band portion is configured to substantially equatorially encircle the eye of the subject; and
an enlarged portion formed along a portion of the length of the band portion, wherein the enlarged portion has a width, wherein the width of the enlarged portion is greater than the width of the band portion, and wherein the enlarged portion and the band portion have a substantially uniform thickness,
wherein the band portion and the enlarged portion are substantially equally compressive, and wherein the band portion and the enlarged portion are configured to be securely positioned against the scleral wall of the eye of the subject such that the enlarged portion overlies and supports the retinal defect.

2. The unitary scleral buckling element of claim 1, wherein the eye of the subject comprises four quadrants of equal length, and wherein the enlarged portion of the scleral buckling element has a length substantially corresponding to the length of one quadrant of the eye of the subject.

3. The unitary scleral buckling element of claim 1, wherein the eye of the subject comprises four quadrants of equal length, and wherein the enlarged portion of the scleral buckling element has a length substantially corresponding to the length of two quadrants of the eye of the subject.

4. The unitary scleral buckling element of claim 1, wherein the eye of the subject comprises four quadrants of equal length, and wherein the enlarged portion of the scleral buckling element has a length substantially corresponding to the length of three quadrants of the eye of the subject.

5. The unitary scleral buckling element of claim 1, wherein the enlarged portion of the scleral buckling element has outer edges that cooperate to define a shape of the enlarged portion, wherein the outer edges of the enlarged portion are substantially smooth.

6. The unitary scleral buckling element of claim 5, wherein the shape of the enlarged portion is substantially circular.

7. The unitary scleral buckling element of claim 5, wherein the shape of the enlarged portion is substantially elliptical.

8. The unitary scleral buckling element of claim 5, wherein the shape of the enlarged portion is substantially rectangular.

9. The unitary scleral buckling element of claim 1, wherein the ratio between the width of the enlarged portion and the width of the band portion ranges from about 1:1 to about 8:1.

10. A method for treating a retinal defect within an eye of a subject, comprising:
substantially equatorially encircling the eye of the subject with a unitary scleral buckling element, the unitary scleral buckling element comprising:
a band portion having a length and a width; and
an enlarged portion formed along a portion of the length of the band portion, wherein the enlarged portion has a width, wherein the width of the enlarged portion is greater than the width of the band portion, and wherein the enlarged portion and the band portion have a substantially uniform thickness; and
securely attaching the scleral buckling element to the scleral wall of the eye of the subject such that the enlarged portion of the scleral buckling element overlies and supports the retinal defect.
